(19) United States
(12) Patent Application Publication
(10) Pub. No.: US 2022/0340914 A1
(43) Pub. Date: Oct. 27, 2022

(54) METHODS OF PREPARING VIRAL VECTORS

(71) Applicant: Repligen Corporation, Waltham, MA (US)

(72) Inventors: Amit Kumar Dutta, Waltham, MA (US); James Ronald Peyser, Billerica, MA (US)

(73) Assignee: Repligen Corporation, Waltham, MA (US)

(21) Appl. No.: 17/782,206

(22) PCT Filed: Dec. 10, 2020
(86) PCT No.: PCT/US2020/064150

§ 371 Date: Jun. 3, 2022

Related U.S. Application Data
(60) Provisional application No. 62/946,082, filed on Dec. 10, 2019.

(51) Int. Cl. C12N 15/64; B01D 61/00; B01D 29/60

(52) U.S. Cl. CPC C12N 15/64; B01D 61/00; B01D 29/603; C12N 2710/10351

(57) ABSTRACT

This disclosure relates generally to process filtration systems, and more particularly to systems utilizing tangential flow filtration.

### PRIORITY

This application claims the benefit of priority under 35 USC § 119 to U.S. Provisional Application Ser. No. 62/946,082, filed Dec. 10, 2019, which is incorporated by reference herein in its entirety and for all purposes.

### FIELD OF DISCLOSURE

This disclosure relates generally to process filtration systems, and more particularly to systems utilizing tangential flow filtration.

### BACKGROUND

Filtration is typically performed to separate, clarify, modify and/or concentrate a fluid solution, mixture or suspension. In the biotechnology and pharmaceutical industries, filtration is vital for the successful production, processing, and testing of new drugs, diagnostics and other biological products. For example, in the process of manufacturing biologicals, using animal or microbial cell culture, filtration is done for clarification, selective removal and concentration of certain constituents from the culture media or to modify the media prior to further processing. Filtration may also be used to enhance productivity by maintaining a culture in perfusion at high cell concentration.

Biologics manufacturing processes have advanced through substantial process intensification. Both eukaryotic and microbial cell culture to produce recombinant proteins, virus-like particles (VLP), gene therapy particles, and vaccines now include cell growth techniques that can achieve 100e⁶ cells/ml or higher. This is achieved using cell retention devices that remove metabolic waste products and refresh the culture with additional nutrients. One of the most common means of cell retention is to perfuse a bioreactor culture using hollow fiber filtration using alternating tangential flow (ATF). Both commercial and development scale processes use a device that controls a diaphragm pump to perform ATF through a hollow fiber filter (see, e.g., U.S. Pat. No. 6,544,424).

Downstream purification of viral vectors is often conducted in batch mode. Batch mode purification may result in lower productivity, variation in product quality, high equipment footprint, and higher production cost. While multicolumn based continuous chromatographic purification of viral vectors has been reported, this method may involve complex valve switching and high chances of process failure. These multi-column based methods also often require expensive resins which increases cost of production.

Precipitation based purification is less expensive than chromatographic purification. Such purification has been previously reported for batch mode, which has all of the previously noted disadvantages.

### SUMMARY

This disclosure describes the use of precipitation for continuous downstream purification of viral vectors. This method is more robust and less expensive than multi-column chromatographic processes.

The present disclosure, in its various aspects, is directed generally to methods of preparation of viral vectors, and related devices and systems. Embodiments according to the present disclosure, including those described herein, may increase particularly the effectiveness and efficiency of processes used for the preparation and purification of viral vectors.

In an aspect, a method of preparation of viral vectors may comprise flowing a solution comprising the viral vectors and an impurity through a system of hollow fiber filters into a feed channel of a tangential flow filtration apparatus. The solution may comprise a salt in an amount sufficient to cause precipitation of the viral vector but not of the impurity. The resulting retentate from the system of hollow fiber filters may be resolubilized. The viral vectors may pass into a permeate after tangential flow filtration.

In various embodiments described here or otherwise, the salt may be calcium phosphate. The step of resolubilizing may comprise adding EDTA saline. The tangential flow filtration may comprise alternating tangential flow filtration or tangential flow depth filtration. The method may comprise flowing the solution through a vessel wherein (a) the vessel mixes the salt into the solution and (b) the vessel is characterized by a narrow distribution of residence times.

In an aspect, a method of purifying viral vectors may comprise flowing a solution comprising the viral vector and an impurity into a feed channel of a tangential flow filtration apparatus. The solution may comprise a salt in an amount sufficient to cause precipitation of the impurity but not of the viral vector. The precipitated impurity may not pass into a permeate while the viral vector may pass into the permeate.

In various embodiments, the retentate may be discarded. The salt may comprise a quaternary ammonium compound. The salt may comprise cetyltrimethylammonium bromide (CTAB). The method may comprise flowing the solution through a vessel wherein (a) the vessel may mix the salt into the solution and (b) the vessel may be characterized by a narrow distribution of residence times. The vessel may be a coiled flow inversion reactor or a stirred tank reactor. The tangential flow filtration apparatus may be an alternating tangential flow (ATF) filtration or tangential flow depth filtration apparatus.

In an aspect, a method of preparation of a viral vector may include flowing a solution comprising the viral vector and an impurity through a first filter comprising a first retentate channel and a first permeate channel. A retentate may be flowed from the first retentate channel of the first filter into a second retentate channel of a tangential flow filtration filter. The retentate may be resolubilized from the first retentate channel of the first filter. The solution may comprise a salt in an amount sufficient to cause substantial precipitation of the viral vector but not of the impurity. The viral vector passes into a second permeate channel of the tangential flow filter.

In various embodiments, the salt may be calcium phosphate. Resolubilizing may further comprise adding EDTA saline to the retentate. The tangential flow filter may comprise an alternating tangential flow (ATF) filter or a tangential flow depth filter. The solution may be flowed through a vessel wherein (a) the vessel mixes the salt into the solution, (b) the vessel is characterized by a narrow distribution of residence times, and (c) the solution is flowed from the vessel towards the first filter. A second filter may be included. The second filter may comprise a third retentate channel in fluid communication with the first retentate channel. The second filter may comprise a third permeate channel in fluid communication with the first retentate channel. A first mixer may be upstream of the first retentate channel. A second mixer may be upstream of the third retentate channel. A buffer may be flowed into the second mixer. The first filter and the second filter may each comprise a flat-sheet cassette, a spiral wound fiber filter, or a hollow fiber filter

In an aspect, a method of concentrating a viral vector may include flowing a solution comprising the viral vector and an impurity into a first retentate channel of a hollow fiber filter. A retentate may be flowed from the first retentate channel of the hollow fiber filter into a second retentate channel of a tangential flow filter. The solution may comprise a salt in an amount sufficient to cause substantial precipitation of the viral vector but not of the impurity. The substantially precipitated impurity may be retained within a second retentate channel of the tangential flow filter. The viral vector may be passed into a permeate channel of the tangential flow filter.

In various embodiments, the salt may be calcium phosphate. The retentate may be resolubilized from the first retentate channel of the first hollow fiber filter by adding EDTA saline to the retentate. The tangential flow filter may comprise an alternating tangential flow (ATF) filter or a tangential flow filter. The solution may be flowed through a vessel wherein (a) the vessel mixes the salt into the solution, (b) the vessel is characterized by a narrow distribution of residence times, and (c) the solution is flowed from the vessel towards the hollow fiber filter.

In an aspect, a method of purifying a viral vector may include flowing a solution comprising the viral vector and an impurity into a feed channel of a tangential flow filter. The solution may comprise a salt in an amount sufficient to cause substantial precipitation of the impurity but not of the viral vector. The substantially precipitated impurity may not pass into a permeate of the tangential flow filter. The viral vector may pass into the permeate of the tangential flow filtration apparatus.

In various embodiments, flowing the solution may comprise the substantially precipitated impurity from the container to a waste. The salt may comprise a quaternary ammonium compound. The salt may comprise cetyltrimethylammonium bromide (CTAB). The solution may be flowed through a vessel wherein (a) the vessel mixes the salt into the solution, (b) the vessel is characterized by a narrow distribution of residence times, and (c) the solution is flowed from the vessel to the container. The vessel may be a coiled flow inversion reactor or a stirred tank reactor. The tangential flow filter may be an alternating tangential flow (ATF) filter or tangential flow filter.

### BRIEF DESCRIPTION OF THE DRAWINGS

FIG. 1 is a schematic illustration of a system for purifying viral vectors, according to an embodiment of the present disclosure.

FIG. 2 is a schematic illustration of a system for concentrating viral vectors, according to an embodiment of the present disclosure.

FIG. 3 is a schematic illustration of a system for continuous purifying viral vectors and precipitating impurities, according to an embodiment of the present disclosure.

### DETAILED DESCRIPTION

### Overview

In precipitation based continuous purification of viral vectors, a reactor and filtration system are used. The reactor may be a continuous stirred tank reactor (CSTR) or a coiled coil reactor (CCR). The filtration system may be operated as an alternating tangential flow (ATF) filter, a tangential flow filter (TFF), or a tangential flow depth filter (TFDF). The method may be used to (i) purify viral vectors, (ii) concentrate viral vectors, or (iii) removing impurities from a viral vector feed. Exemplary filters may include hollow fiber filters having, e.g., pore sizes ranging from about 1 kda to about 15 μm for TFDF operation or larger pore sizes for a TFDF filter, operated in one or both TFF or ATF mode. In various embodiments described herein, a TFF operating in ATF mode may have less fouling (compared to non-ATF) due to changes in flow direction within the retentate channel along the filter. This may increase filter performance. In various embodiments described herein, TFDF may allow for faster flow rate but it may have lower filtration capacity than TFF or ATF.

In certain embodiments, solutions are mixed and the resulting material flows through the system via gravity, induced pressure (e.g., a mag-lev, peristaltic or diaphragm/piston pump), or other forces. The material moves through the system at a rate dependent on precipitation kinetics of either the product or the impurities present. Once material arrives at the filtration system containing an ATF, TFF, TFDF, or the like, a pressure system impels the material through the filtration system. In some embodiments, the pressure system may include a diaphragm pump.

In certain embodiments, the likely impurities may consist of host cell proteins and nutrients used in the feed medium.

In certain embodiments, the system contains a reactor, e.g., a coiled coil reactor, i.e., a coiled flow inversion reactor, or a continuous stirred tank reactor. Without wishing to be bound by any theory, it is believed that a coiled flow inversion reactor acts to enhance radial mixing, creating a narrow residence time distribution. The use of a coiled coil reactor or a continuous stirred tank reactor may depend on precipitation kinetics. In some embodiments, the mixed material would flow into a series of static mixers and hollow fiber filters in order to remove impurities. The membrane pore size may vary and may depend on the size of the viral vector and precipitates present in the system. Waste is removed from the system and buffer added while the material is flowing through the series of static mixers and hollow fiber filters. The resulting retentate of such a system contains the precipitate, which is resolubilized before flowing through a filtration system. Portions of the filtration system may comprise ATF, TFF, or TFDF operation and may include a hollow fiber, flat sheet cassette filter, or spiral wound fiber filter.

In certain embodiments, the system contains a reactor, e.g., a coiled coil reactor, i.e., a coiled flow inversion reactor, or a continuous stirred tank reactor. A viral vector is precipitated in such a reactor, and the resulting mixture flowed through a hollow fiber filter. The resulting retentate contains the precipitate and may be resolublized to be flowed through a filtration system. Portions of the filtration system may comprise ATF, TFF, or TFDF operation and may include a hollow fiber, flat sheet cassette filter, or spiral wound fiber filter.

In certain embodiments, the system contains a reactor, e.g., a coiled coil reactor, i.e., a coiled flow inversion reactor, or a continuous stirred tank reactor. A solution containing impurities is mixed in said reactor, precipitating the impurities. The resulting mixture has the precipitated impurities removed from the system and the resulting solution flowed through a filtration system. Portions of the filtration system may comprise ATF, TFF, or TFDF operation and may include a hollow fiber, flat sheet cassette filter, or spiral wound fiber filter.

In certain embodiments, the system is used for proteins, nanoparticles, and viruses (e.g., AAV, lentivirus; virus-like particles, microparticles, microcarriers, microspheres, nanoparticles, and the like).

In certain embodiments, the viral vector is precipitated. Without wishing to be bound by any theory, precipitating viral vectors is believed to allow for the removal of the viral vector from the solution via filtration, with the precipitated viral vector in the retentate. This method is used for purification of viral vectors, concentration of viral vectors, or similar processes.

In some embodiments, impurities are precipitated. The precipitated impurities are then removed from the mixture, and the resulting solution flowed through a filtration system.

In some embodiments, an impure viral vector is mixed with a precipitating agent (i.e., calcium phosphate, ammonium sulfate) within a bioreactor, specifically a coiled coil reactor or a continuous stirred tank reactor. The precipitating agent specifically precipitates the viral vector. The solution is flowed through a series of static mixers and hollow fiber filters. Without wishing to be bound by any theory, this series is used in order to increase both precipitation of the viral vectors and removal of those viral vectors from the system. The retentate containing the precipitate is collected from the filters and a solution (i.e., 0.1 M EDTA saline) added in order to resolubilize the viral vectors. The resolubilized solution is filtered in order to produce pure viral vectors.

In some embodiments, a dilute viral vector is mixed with a precipitating agent (i.e., calcium phosphate) within a reactor, specifically a coiled coil reactor or a continuous stirred tank reactor. The precipitating agent specifically precipitates the viral vector. The solution is flowed through a hollow fiber filter. The resulting retentate contains the precipitated viral vector, and the resulting permeate is removed as waste. The precipitate is resolubilized and filtered, resulting in a concentrated viral vector.

In certain embodiments, an impure viral vector is mixed with a precipitating agent (i.e., cetyl trimethyl ammonium bromide (CTAB), domiphen bromide, or the like) within a reactor, specifically a coiled coil reactor or a continuous stirred tank reactor. The precipitating agent specifically precipitates the impurities in the solution. After mixing, the impurities are removed from the mixture, wherein the solution containing the viral vectors is filtered, resulting in purified product.

In certain embodiments, further downstream processing may be necessary to remove trace amounts of impurities. In some embodiments, the cell culture fluid should be clarified prior to use in the described system. If connected to a continuous clarification system, the upstream bioreactor can be directly integrated into the described system.

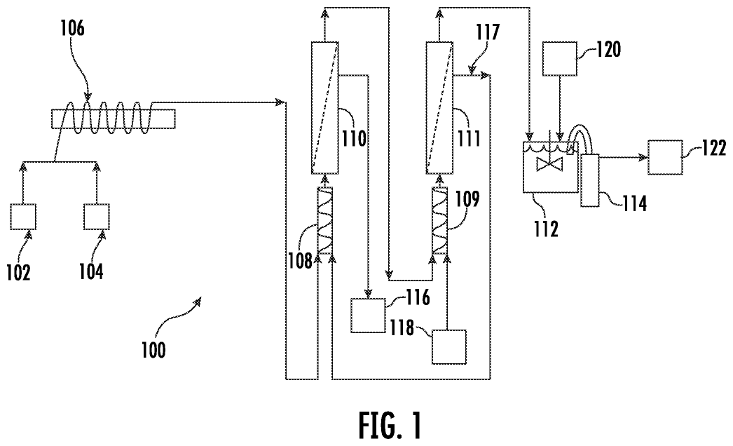

FIG. 1 illustrates an exemplary system for preparing and purifying a viral vector. The system 100 includes a reactor 106, e.g., a coiled coil reactor, which connects to a system of first and second mixers 108, 109 and first and second hollow fiber filters 110, 111 (e.g., a combination of a hollow fiber and a mixer in series may be referred to as a “stage” that may be operated in ATF or TFF). Although two stages are illustrated, any number of stages may be used (e.g., 0, 1, 2, 3, 4, 10, etc.). The number of stages to be used will depend on yield requirement. Increase in number of stages increases product yield but it increases system cost as well. An impure viral vector 102 and a salt 104 (e.g., calcium phosphate, ammonium sulfate, another precipitating agent, or the like) are added to the reactor 106 to form and/or mix into a solution for flowing through the system 100. The solution is flowable from the reactor 106 to a first mixer 108 positioned upstream of the first hollow fiber filter 110. The first mixer 108 is configured to mix the solution with a downstream permeate (as discussed below). The product of the first mixer 108 is flowable into the first hollow fiber filter 110. The first hollow fiber 110 filters off some impurities through a first permeate channel 116 into a waste. A first retentate channel of the first hollow fiber filter 110 is in fluid communication with a second mixer 109 positioned upstream of the second hollow fiber filter 111. The second mixer 109 is configured to mix the retentate from the first retentate channel with a buffer 118 to assist with precipitating the viral vector that is added to the second mixer 109. The product of the second mixer 109 is flowable into the second hollow fiber filter 111. The second hollow fiber filter 111 filters off some impurities (e.g., undesired species) and non-precipitated viral vector through a second permeate channel 117 that is flowable to the first mixer 108 for further processing as mentioned above. In various embodiments, a pore size of the filters may depend on a particle size of the precipitate and the product. A ratio of buffer flow rate to inlet feed flow rate may depend on a desired product yield. Increasing the ratio of buffer flow rate to inlet feed flow rate may increase the product yield but may require additional buffer and may dilute the product. A second retentate channel of the second hollow fiber filter 111 is in fluid communication with a container 112 such that the product of the second retentate channel is flowable into the container 112. The container 112 containing the precipitated viral vector may be substantially resolubilized into a solution by adding a saline 120 (e.g., about 0.1 M EDTA saline, or the like). The resolubilized solution within the container 112 is flowable through a third filter 114 (e.g., a filter in ATF, TFF, TFDF operation). The third filter 114 filters out a substantially purified viral vector through a third permeate channel 122.

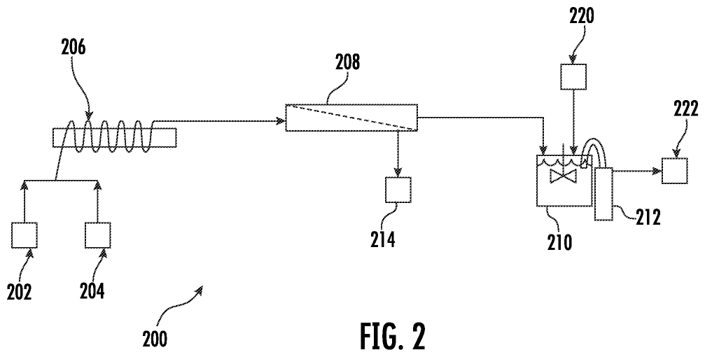

FIG. 2 illustrates an exemplary system for concentrating a viral vector. The system 200 includes a reactor 206, e.g., a coiled coil reactor, which connects to a hollow fiber filter 208. Although one hollow fiber filter 208 is illustrated, any number of filters may be used (e.g., 2, 3, 4, 10 etc.). A dilute viral vector 202 and a salt 204 (e.g., calcium phosphate, ammonium sulfate, another precipitating agent, or the like) are added to the reactor 206 to form and/or mix into a solution for flowing through the system 200. The solution is flowable from the reactor 206 to a hollow fiber filter 208. The hollow fiber filter 208 filters off some impurities and non-precipitated viral vector (e.g., undesired species) through a permeate channel 214 that is flowable to a waste. A first retentate channel of the hollow fiber filter 208 is in fluid communication with a container 210 such that substantially precipitated viral vector is flowable from the first retentate channel to the container 210. The container 210 containing the precipitated viral vector may be resolubilized into a solution by adding a saline 220 (e.g., about 0.1 M EDTA saline, or the like). The resolubilized solution within the container 210 is flowable through a tangential filter 212 (e.g., operated in a ATF mode, TFF mode, TFDF mode, or the like). The tangential filter 212 filters out a substantially concentrated viral vector through a second permeate channel 222. The tangential filter 212 may operate continuously to produce the concentrated viral vector through the second permeate channel 222 without adding further fluid to the container 210 because the retentate of the tangential filter 212 may reciprocate flow between the container 210 and the tangential filter 212. In this way, the tangential filter 212 may continue to amplify the concentrated viral vector produced from the second permeate channel 222 without further processing steps and/or equipment.

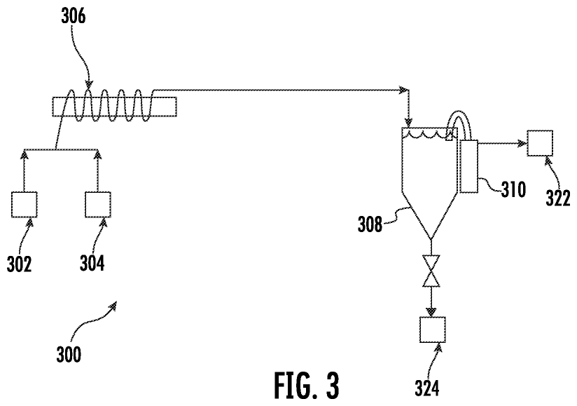

FIG. 3 illustrates an exemplary system for precipitating impurities in a solution and purifying a viral vector of a solution. The system 300 comprises a reactor 306, e.g., a coiled coil reactor. Although no hollow fiber filter (as described herein) is illustrated, any number of filters may be used in-line with the reactor 306 (e.g., 1, 2, 3, 4, 5, 6, 8, 10, 15, 20, 50, 100, etc.). An impure viral vector 302 (e.g., adeno-associated virus (AAV) vectors) and a salt 304 (e.g., CTAB, domiphen bromide, another precipitating agent, or the like) are added to the reactor 306 to form and/or mix into a solution for flowing through the system 300. Impurities (e.g., undesirable materials) of the solution are substantially precipitated within the reactor 306 and the mixed solution is flowable from the reactor 306 to a container 308. The container 308 containing the precipitated impurities is flowable through a tangential filter 310 (e.g., an ATF, TFF, TFDF, or the like). The tangential filter 310 filters out a substantially purified viral vector through a permeate channel 322. The precipitated impurities are retained within a retentate channel of the tangential filter 310 and are maintained or returned to the container 308. The container 308 includes a waste channel 324 to receive (e.g., “bleed”) the precipitated impurities from the container 308. The waste channel 324 may be flowed using a pump, gravity, a metered valve, a timed valve, a manual valve, an open flow path, a restricted flow path, a filter, a combination thereof, or the like. The tangential filter 310 may operate continuously to produce the purified viral vector through the permeate channel 322 because the retentate of the tangential filter 310 may reciprocate flow between the container 308 and the tangential filter 310. As precipitated impurities are flowed from the reactor 306 into the container 308, precipitated impurities are further flowed from the container 308 into the waste channel 324. Therefore, a substantially consistent volume of fluid may be maintained in the container 308 such that the filter 310 is not overburdened, does not run out of fluid to filter, and maintains a substantially consistent mass flowrate. A ratio of the flowrate from the reactor 306 to the container 308, the flowrate of the precipitated impurities into the waste channel 324, and the flowrate of the fluid from the container 308 into the retentate of the filter 310 may be arranged such that continuous operation of the system 300 producing purified viral vector through the permeate channel 322 is maintained without further processing steps and/or equipment.

### CONCLUSION

The foregoing disclosure has presented several exemplary embodiments of filtration systems according to the present disclosure. These embodiments are not intended to be limiting, and it will be readily appreciated by those of skill in the art that various additions or modifications may be made to the systems and methods described above without departing from the spirit and scope of the disclosure. Additionally, while the foregoing disclosure has focused primarily on alternating tangential flow filtration systems and their applications, it will be appreciated by those of skill in the art that the principles of the disclosure are applicable to other systems including hollow-fiber TFF and TFDF and other filtration systems.

1. A method of preparation of a viral vector, comprising:
flowing a solution comprising the viral vector and an impurity through a first filter comprising a first retentate channel and a first permeate channel;
flowing a retentate from the first retentate channel of the first filter into a second retentate channel of a tangential flow filtration filter; and
resolubilizing the retentate from the first retentate channel of the first filter;
wherein the solution comprises a salt in an amount sufficient to cause substantial precipitation of the viral vector but not of the impurity; and
wherein the viral vector passes into a second permeate channel of the tangential flow filter.

2. The method of claim 1, wherein the salt is calcium phosphate.

3. The method of claim 1, wherein resolubilizing further comprises adding EDTA saline to the retentate.

4. The method of claim 1, wherein the tangential flow filter comprises an alternating tangential flow (ATF) filter or a tangential flow depth filter.

5. The method of claim 1, further comprising flowing the solution through a vessel wherein (a) the vessel mixes the salt into the solution, (b) the vessel is characterized by a narrow distribution of residence times, and (c) the solution is flowed from the vessel towards the first filter.

6. The method of claim 1, further comprising a second filter, the second filter comprising a third retentate channel in fluid communication with the first retentate channel, and the second filter comprising a third permeate channel in fluid communication with the first retentate channel.

7. The method of claim 6, further comprising a first mixer upstream of the first retentate channel and a second mixer upstream of the third retentate channel.

8. The method of claim 6, wherein the first filter and the second filter each comprise a flat-sheet cassette, a spiral wound fiber filter, or a hollow fiber filter.

9. A method of concentrating a viral vector, comprising:
flowing a solution comprising the viral vector and an impurity into a first retentate channel of a hollow fiber filter; and
flowing a retentate from the first retentate channel of the hollow fiber filter into a second retentate channel of a tangential flow filter;
wherein the solution comprises a salt in an amount sufficient to cause substantial precipitation of the viral vector but not of the impurity;
wherein the substantially precipitated impurity is retained within a second retentate channel of the tangential flow filter; and
wherein the viral vector passes into a permeate channel of the tangential flow filter.

10. The method of claim 9, wherein the salt is calcium phosphate.

11. The method of claim 9, further comprising resolubilizing the retentate from the first retentate channel of the first hollow fiber filter by adding EDTA saline to the retentate.

12. The method of claim 9, wherein the tangential flow filter comprises an alternating tangential flow (ATF) filter or a tangential flow filter.

13. The method of claim 9, further comprising flowing the solution through a vessel wherein (a) the vessel mixes the salt into the solution, (b) the vessel is characterized by a narrow distribution of residence times, and (c) the solution is flowed from the vessel towards the hollow fiber filter.

14. A method of purifying a viral vector, comprising:
flowing a solution comprising the viral vector and an impurity into a feed channel of a tangential flow filter;
wherein the solution comprises a salt in an amount sufficient to cause substantial precipitation of the impurity but not of the viral vector;
wherein the substantially precipitated impurity does not pass into a permeate of the tangential flow filter; and
wherein the viral vector passes into the permeate of the tangential flow filtration apparatus.

15. The method of claim 14, further comprising flowing the solution comprising the substantially precipitated impurity from the container to a waste.

16. The method of claim 14, wherein the salt comprises a quaternary ammonium compound.

17. The method of claim 14, wherein the salt comprises cetyltrimethylammonium bromide (CTAB).

18. The method of claim 14, further comprising flowing the solution through a vessel wherein (a) the vessel mixes the salt into the solution, (b) the vessel is characterized by a narrow distribution of residence times, and (c) the solution is flowed from the vessel to the container.

19. The method of claim 18, wherein the vessel is a coiled flow inversion reactor or a stirred tank reactor.

20. The method of claim 14, wherein the tangential flow filter is an alternating tangential flow (ATF) filter or tangential flow filter.
